(19) United States
(12) Patent Application Publication
(10) Pub. No.: US 2013/0324863 A1
(43) Pub. Date: Dec. 5, 2013

(54) GUIDE WIRE ARRANGEMENT, STRIP ARRANGEMENT AND METHODS OF FORMING THE SAME

(71) Applicants: Daquan Yu, Singapore (SG); Woo Tae Park, Singapore (SG); Li Shiah Lim, Singapore (SG); Muhammad Hamidullah, Singapore (SG); Rama Krishna Kotlanka, Singapore (SG); Vaidyanathan Kripesh, Singapore (SG); Hanhua Feng, Singapore (SG)

(72) Inventors: Daquan Yu, Singapore (SG); Woo Tae Park, Singapore (SG); Li Shiah Lim, Singapore (SG); Muhammad Hamidullah, Singapore (SG); Rama Krishna Kotlanka, Singapore (SG); Vaidyanathan Kripesh, Singapore (SG); Hanhua Feng, Singapore (SG)

(21) Appl. No.: 13/883,274

(22) PCT Filed: Nov. 2, 2011
(86) PCT No.: PCT/SG11/00389

§ 371 Date: Aug. 16, 2013

(30) Foreign Application Priority Data
Nov. 3, 2010 (SG) 201008115-6

(51) Int. Cl. A61B 5/0215

(52) U.S. Cl. CPC A61B 5/02158
USPC 600/486; 29/854

(57) ABSTRACT

A guide wire arrangement, a strip arrangement, a method of forming a guide wire arrangement, and a method of forming a strip arrangement are provided. The guide wire arrangement includes a strip; a sensor being disposed on a first portion of the strip; a chip being disposed next to the sensor on a second portion of the strip, wherein the second portion of the strip is next to the first portion of the strip; wherein the strip is folded at a folding point between the first portion of the strip and the second portion of the strip such that the first portion of the strip and the second portion of the strip form a stack of strip portions.

### TECHNICAL FIELD

Various embodiments relate generally to a guide wire arrangement, a method of forming a guide wire arrangement, a strip arrangement, and a method of forming a strip arrangement.

### BACKGROUND

Minimally invasive surgical procedures are generally preferred because of small incisions which leave small tissue scar after healing, shorter hospitalization time and faster recovery from incision trauma. For many cardio vascular and thoracic interventional procedures, passing a guide wire through a vascular vessel is usually the first step followed by surgical procedures such as stenting. Multiple and prolonged attempts at guide wire passage might lead to several undesirable side-effects such as increased exposure to radiation dosage (fluoroscopy time), increased amounts of intravenous contrast used resulting in an increased risk of nephrotoxicity with consequent renal failure, and increased risk of developing intravascular complications from aggressive guide wire manipulation.

The step of passing the guide wire through a vascular vessel may be primarily through the haptic feeling of the surgeon, and tactile/force feedback of the passing guide wire may be difficult to quantify. The tactile/force feedback of the passing guide wire may be important and useful for comparative evaluation of the surgical procedure e.g. either by residents or by senior surgeons for training the residents. The existing methods for guide wire passage may be heavily dependent on two dimensional fluoroscopic x-ray imaging that is extra-luminal in nature. That is, the vessels are visualized in two planes externally via x-rays and intravenous contrast. There may also be a significant amount of dependence on hand-eye co-ordination between the surgeon, the on-screen x-ray images and on tactile feedback during wire/catheter manipulation. This may result in a series of complex steps requiring focused movements on the surgeon's part.

Microelectromechanical systems (MEMS) have enabled the possibility of making sensorized guide wires. Yoichi Haga et al [1] describes placing a pressure sensor at the tip of the guide wire so that information pertaining to the exact location of the stenosis can be obtained by the difference in the pressure at the lesion, thus reducing the intravenous contrast usage and minimizing the risk of possible renal failures. Keith et al [2] describes that there is a change of about 3° C. in temperature at the location of the stenosis. Hence, a temperature sensor is used at the tip of the guide wire. Gianluca et al [3] describes that the hardness of the calcified tissue at the stenosis location is higher than the healthy vascular vessel. Thus, a force sensor can be used to identify stenosis.

For the guide wire to be passed through a vascular vessel, the length of the guide wire is preferably as long as possible and the diameter of the guide wire is preferably as small as possible. However, the packaging and integration of such long guide wire with very small devices (e.g. sub-millimeter devices such as MEMS and ASIC) pose a challenge.

U.S. Pat. No. 7,162,926 B1 describes a ceramic substrate including embedded connectors used to hold a MEMS sensor. The embedded connectors are in contact with the sealed cavity and are also in contact with the electrical circuit embedded into the body to pass electrical signals from the MEMS sensor to the electrical circuit. However, high costs may be incurred to form such a structure and the fabrication and assembly process may be complex. Further, it may also be difficult to apply such a structure in a guide wire with small diameter.

U.S. Pat. No. 6,106,486 describes a method of manufacturing a conductor element for a guide wire with conductors in the form of a conductive material extending along the length of the conductor element. U.S. Pat. No. 6,106,486 also describes a guide wire which has one core element and overlapping layers of alternating insulating and conductive materials were applied concentrically around the circumference of the core element along a portion of its length, until a desired number of conductive layers have been applied. However, it may be difficult to make such patterns on a long guide wire with small diameter. There may also be less flexibility of sensor placement direction. Further, for core element with a small diameter, the layer of conductive materials deposited on the core element may be thin. As such, the resistance value of the conductive materials may be very high. In addition, bonding of the exposed conductive materials on the core element and small MEMS device may also be difficult.

U.S. Pat. No. 6,090,052 describes a guide wire including a core wire having a proximal and a distal end. There is at least one electrical lead provided on the core wire. The electrical lead extends along the length of the core wire and is connected to an electrical device provided at the dismal end of the core wire. A male connector is provided at the proximal end of the core wire, and a protective tubing covers the core wire and the electrical leads. The electrical leads are formed on a sheet of a thin flexible material. The sheet of thin flexible material is at least partially wrapped around the core wire along the length of the core wire. The core wire is thus used to house devices and attach electrical leads on it to transmit the electrical signals. The core wire also provides mechanical support for operation. However, for a guide wire with a small diameter, electrical leads provided on the core wire may be thin. This may result in high resistance of the electrical leads. The high resistance of the electrical leads may lead to signal retard or even wrong information received by terminal side. Further, the small components may need to be assembled under microscope, which is very tedious and labor intensive.

### SUMMARY

According to one embodiment, a guide wire arrangement is provided. The guide wire arrangement includes a strip; a sensor being disposed on a first portion of the strip; a chip being disposed next to the sensor on a second portion of the strip, wherein the second portion of the strip is next to the first portion of the strip; wherein the strip is folded at a folding point between the first portion of the strip and the second portion of the strip such that the first portion of the strip and the second portion of the strip form a stack of strip portions.

According to another embodiment, a method of forming a guide wire arrangement is provided. The method includes providing a strip; disposing a sensor on a first portion of the strip; disposing a chip next to the sensor on a second portion of the strip, wherein the second portion of the strip is next to the first portion of the strip; folding the strip at a folding point between the first portion of the strip and the second portion of the strip such that the first portion of the strip and the second portion of the strip form a stack of strip portions.

According to yet another embodiment, a strip arrangement is provided. The strip arrangement includes a strip having a first surface and a second surface; at least one first wire being disposed on the second surface of the strip and electrically connected to the strip via a through-hole formed in the strip; at least one second wire being disposed on the first surface of the strip and electrically connected to the strip.

According to another embodiment, a method of forming a strip arrangement is provided. The method includes providing a strip having a first surface and a second surface; disposing at least one first wire on the second surface of the strip and electrically connecting the at least one first wire to the strip via a through-hole formed in the strip; disposing at least one second wire on the first surface of the strip and electrically connecting the at least one second wire to the strip.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1a shows a schematic top view of a guide wire arrangement according to one embodiment.

FIG. 1b shows a schematic side view of a guide wire arrangement according to one embodiment.

FIG. 1c shows an image of a first surface of a strip of a guide wire arrangement before the strip is folded according to one embodiment.

FIG. 1d shows an image of a second surface of a strip of a guide wire arrangement before the strip is folded according to one embodiment.

FIG. 1e shows a schematic top view of a guide wire arrangement after a strip of the guide wire arrangement is folded at a folding point according to one embodiment.

FIG. 1f shows a schematic side view of a guide wire arrangement after a strip of the guide wire arrangement is folded at a folding point according to one embodiment.

FIG. 1g shows an image of a first surface of a strip of a guide wire arrangement after the strip is folded at a folding point according to one embodiment.

FIG. 1h shows an image of a second surface of a strip of a guide wire arrangement after the strip is folded at a folding point according to one embodiment.

FIG. 1i shows a schematic top view of a guide wire arrangement after a strip of the guide wire arrangement is folded at a further folding point according to one embodiment.

FIG. 1j shows a schematic side view of a guide wire arrangement after a strip of the guide wire arrangement is folded at a further folding point according to one embodiment.

FIG. 2 shows a process of forming a strip of a guide wire arrangement according to one embodiment.

FIG. 3 shows a schematic top view of a strip of a guide wire arrangement according to one embodiment.

FIG. 4 shows images of a top view of a strip of a guide wire arrangement according to one embodiment.

FIG. 5 shows that a schematic diagram of a guide wire arrangement having a sensor, a chip and wires disposed a strip of the guide wire arrangement according to one embodiment.

FIG. 6 shows images of a solder bump formed on a test chip.

FIG. 7a shows an image of two wires disposed on a first surface of a strip of a guide wire arrangement according to one embodiment.

FIG. 7b shows an image of a wire disposed on a second surface of a strip of a guide wire arrangement according to one embodiment.

FIG. 8 shows a guide wire arrangement 100 having fixtures/holders formed on a first surface and a second surface of a strip of the guide wire arrangement according to one embodiment.

FIG. 9 shows a guide wire arrangement including a housing according to one embodiment.

FIG. 10 shows an image of a guide wire arrangement according to one embodiment.

FIG. 11 shows an exemplary assembly process of arranging a sensor and a chip in a stack on a strip of a guide wire arrangement according to one embodiment.

FIG. 12 shows an image of a top view of an arrangement having two dummy chips bonded respectively on a first surface and a second surface of a strip of a guide wire arrangement according to one embodiment.

FIG. 13 shows a flowchart of a method of forming a guide wire arrangement according to one embodiment.

FIG. 14 shows a schematic diagram of a strip arrangement according to one embodiment.

FIG. 15 shows a flowchart of a method of forming a strip arrangement according to one embodiment.

### DETAILED DESCRIPTION

Embodiments of a guide wire arrangement, a method of forming a guide wire arrangement, a strip arrangement, and a method of forming a strip arrangement will be described in detail below with reference to the accompanying figures. It will be appreciated that the embodiments described below can be modified in various aspects without changing the essence of the invention.

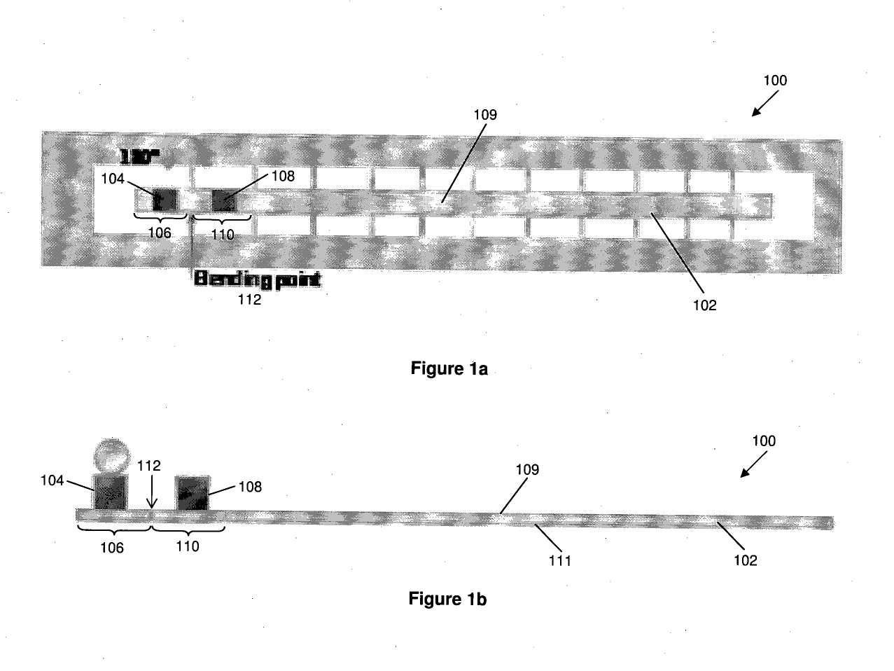

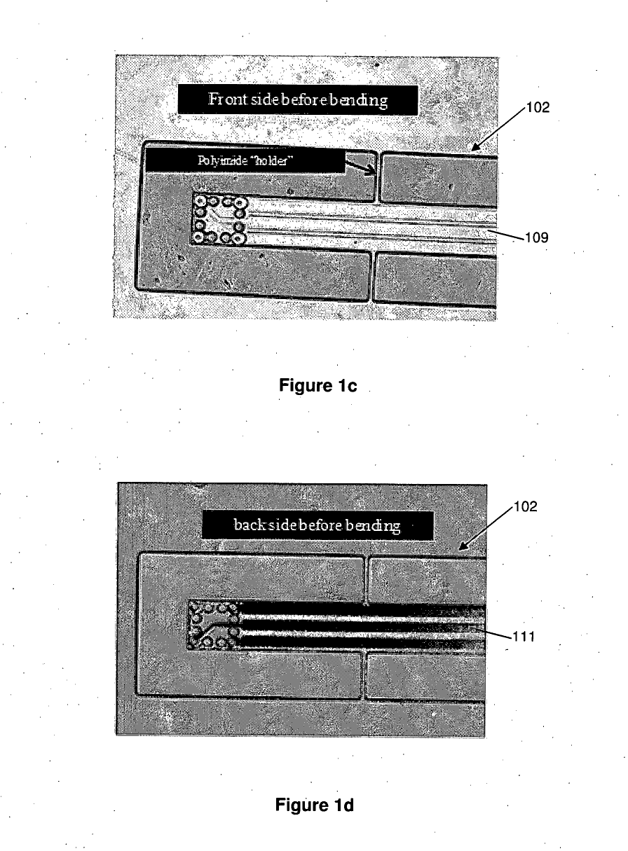

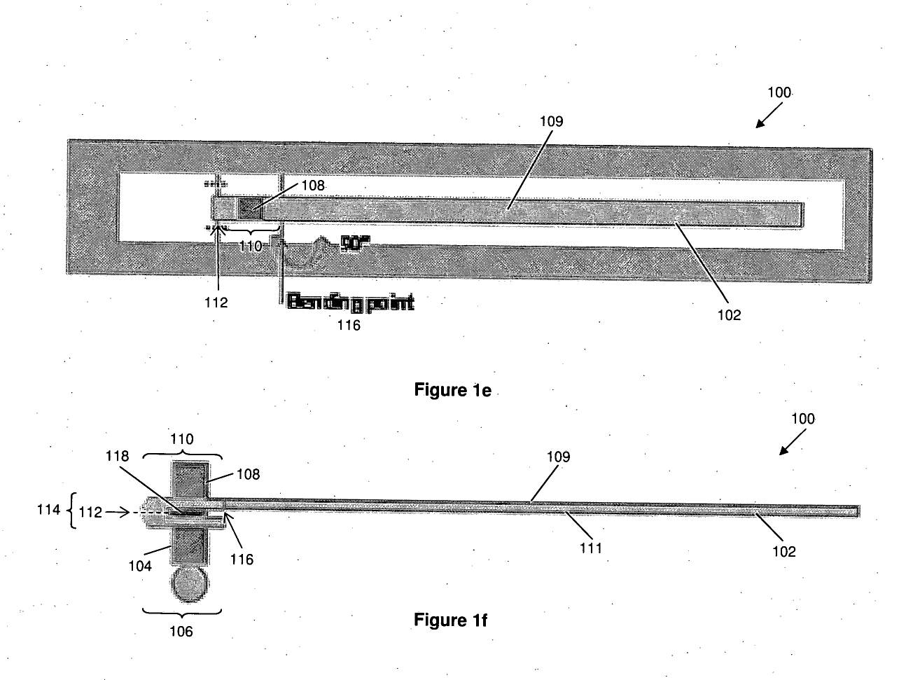

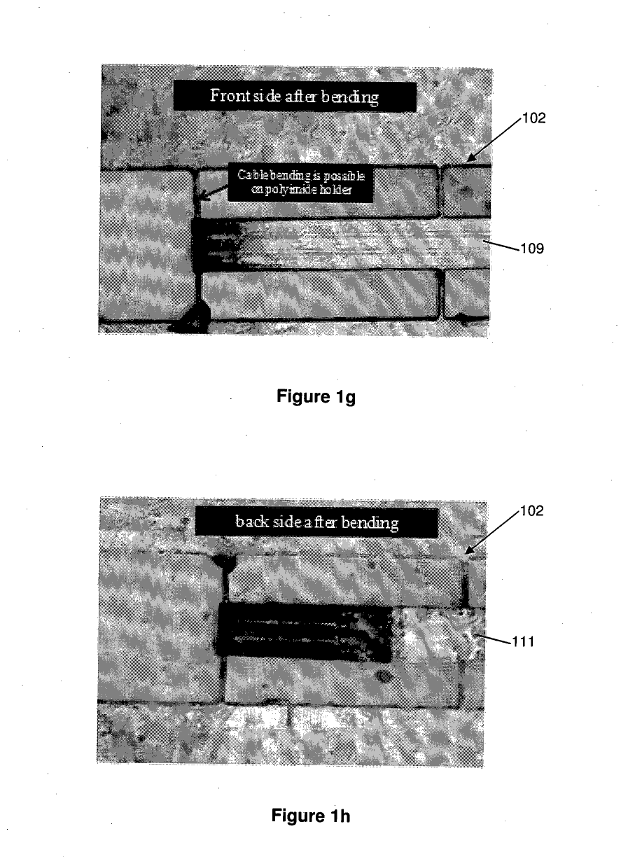

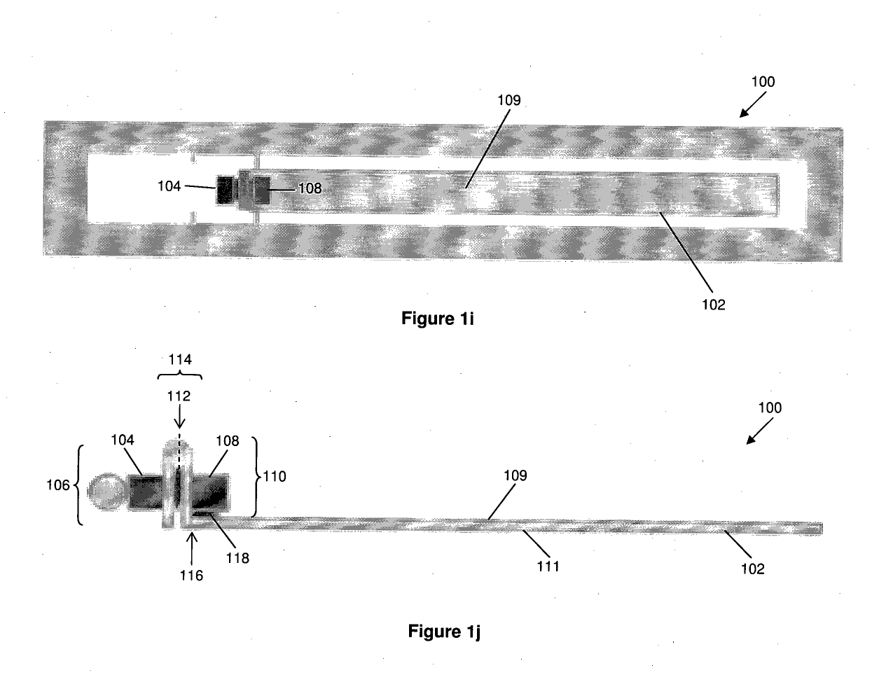

FIG. 1a shows a schematic top view of a guide wire arrangement 100. FIG. 1b shows a schematic side view of the guide wire arrangement 100. The guide wire arrangement 100 includes a strip 102. The strip 102 may be a cable. The guide wire arrangement 100 also includes a sensor 104 disposed on a first portion 106 of the strip 102, and a chip 108 disposed next to the sensor on a second portion 110 of the strip 102. The second portion 110 of the strip 102 is next to the first portion 106 of the strip 102. The strip 102 has a first surface 109 and a second surface 111. The sensor 104 and the chip 108 may be disposed on the first surface 109 of the strip 102. The strip 102 may have a folding point 112 along which the strip 102 can be folded.

In one embodiment, the sensor 104 may be a microelectromechanical system sensor. In another embodiment, the sensor 104 may be a force sensor, a pressure sensor, a temperature sensor, an acceleration sensor, an angular velocity sensor, an electronic compass, or an ultrasound sensor.

In one embodiment, the chip 108 may be an application-specific integrated circuit (ASIC).

FIG. 1c shows an image of the first surface 109 of the strip 102 (without the sensor 104 and the chip 108) before the strip 102 is folded. FIG. 1d shows an image of the second surface 111 of the strip 102 before the strip 102 is folded.

FIG. 1e shows a schematic top view of the guide wire arrangement 100 after the strip 102 is folded at the folding point 112. FIG. 1f shows a schematic side view of the guide wire arrangement 100 after the strip 102 is folded at the folding point 112. The strip 102 is folded at the folding point 112 between the first portion 106 of the strip 102 and the second portion 110 of the strip 102 such that the first portion 106 of the strip 102 and the second portion 110 of the strip 102 form a stack 114 of strip portions. The strip 102 is folded at the folding point 112 such that the sensor 104 faces away the second portion 110 of the strip 102 and the chip 108 faces away from the first portion 106 of the strip 102. The strip 102 is folded at the folding point 112 by about 175 degrees to about 185 degrees. The folded areas (e.g. the first portion 106 and the second portion 110) of the strip 102 are secured using e.g. epoxy 118. The epoxy 118 may be biocompatible.

The strip 102 may have a further folding point 116 along which the strip 102 may be further folded.

FIG. 1g shows an image of the first surface 109 of the strip 102 (without the sensor 104 and the chip 108) after the strip 102 is folded at the folding point 112. FIG. 1h shows an image of the second surface 111 of the strip 102 (without the sensor 104 and the chip 108) after the strip 102 is folded at the folding point 112.

FIG. 1i shows a schematic top view of the guide wire arrangement 100 after the strip 102 is folded at the further folding point 116. FIG. 1j shows a schematic side view of the guide wire arrangement 100 after the strip 102 is folded at the further folding point 116. The strip 102 is folded at the further folding point 116 next to the chip 108 and at the opposite side of the chip 108 than the folding point 112 such that the stack 114 of strip portions 106, 110 is between the folding point 112 and the further folding point 116. The strip 102 is folded at the further folding point 116 by about 85 degrees to about 95 degrees. To keep the guide wire arrangement 100 in the configuration as shown in FIG. 1c, the chip 108 may be secured to the strip 102 using e.g. epoxy 118. The epoxy 118 may be biocompatible.

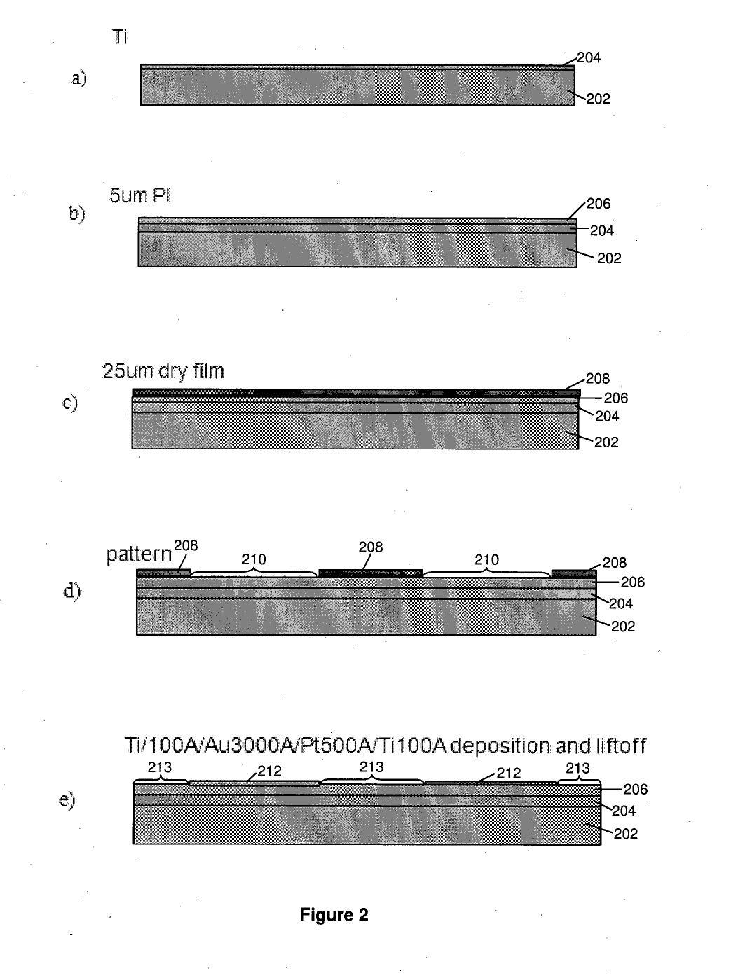

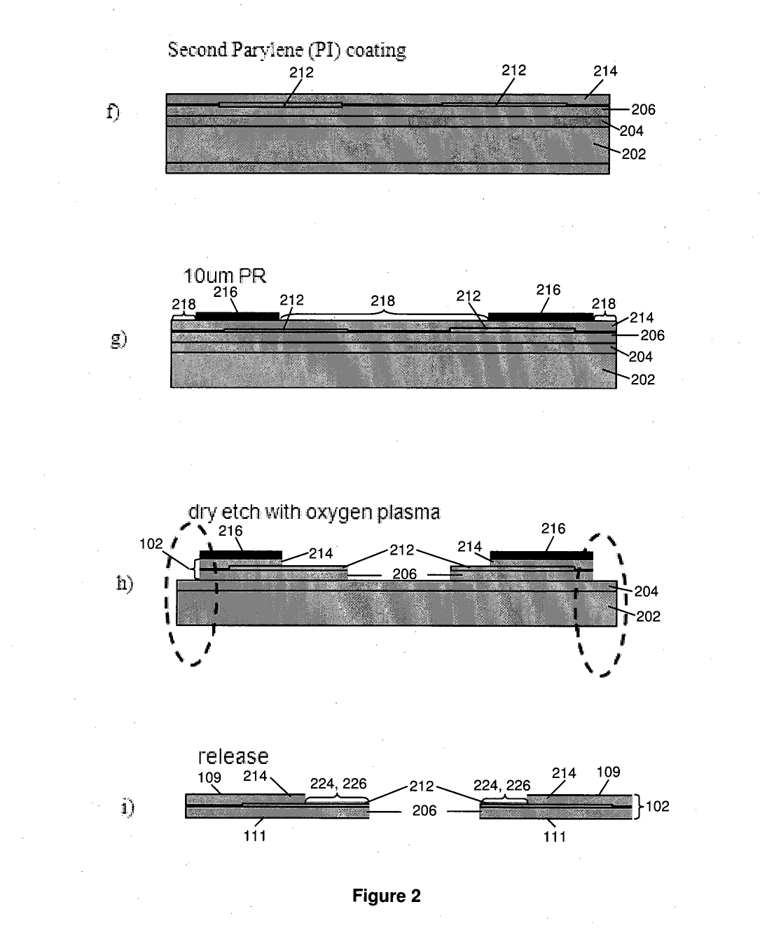

FIG. 2 shows a process of forming the strip 102 of the guide wire arrangement 100. FIG. 2a shows a layer of titanium 204 disposed on a substrate 202. FIG. 2b shows an isolation layer 206 disposed on the layer of titanium 204. The isolation layer 206 may be about 5 μm thick. Various materials may be used for the isolation layer 206. One example may be polyimide. FIG. 2c shows a negative photoresist 208 disposed on the first isolation layer 206. FIG. 2d shows that the negative photoresist 208 is patterned to expose portions 210 of the first isolation layer 206. The negative photoresist 208 may be patterned by applying a lift-off method. FIG. 2e shows that metal is disposed on the exposed portions 210 of the isolation layer 206, forming metal layer 212. The negative photoresist 208 is removed to expose portions 213 of the isolation layer 206. Various materials may be used for the metal layer 212. The metal layer 212 may include any one or more of titanium, gold and platinum. The metal layer 212 may be formed by sputtering and may be used for metallization of interconnection pads, connecting lines and electrode sites. FIG. 2f shows that a further isolation layer 214 is disposed on the metal layer 212 and the isolation layer 206. Various materials may be used for the further isolation layer 214. One example may be polyimide. The further isolation layer 214 may be used for insulation purpose. FIG. 2g shows that a photoresist layer 216 is disposed on the further isolation layer 214 and is patterned to expose portions 218 of the further isolation layer 214. FIG. 2h shows that the isolation layer 206, the metal layer 212 and the further isolation layer 214 are patterned and dry etched to form the strip 102. The isolation layer 206, the metal layer 212 and the further isolation layer 214 may be patterned and dry etched using oxygen plasma. Oxygen plasma can be used to achieve structures with steep edges and low surface roughness such that electrode sites and connection pads may be exposed and devices may be detached from the wafer by etching. FIG. 2i shows that the photoresist layer 216 is removed and the strip 102 is removed from the layer of titanium 204 and the substrate 202. The strip 102 may be removed manually using tweezers. The strip 102 may be a polyimide (PI) substrate embedded with the metallization layers. The strip 102 may be highly flexible and bendable, and may have a thickness of about 10 μm.

A multi-layer process of incorporating metal tracks, micro vias and Micro Flex interconnections is described above with reference to FIG. 2. The process can form highly flexible and ultra-thin substrates where metal microelectrodes, interconnection pads and conducting tracks are placed. The polyimide (PI) substrate (i.e. the strip 102) can allow interconnection of silicon chips and surface mount devices (SMDs) on the polyimide (PI) substrate.

Referring to FIG. 2i, the strip 102 includes a first isolation layer 214 providing a first surface 109 of the strip 102, and a second isolation layer 206 providing a second surface 111 of the strip 102. The strip 102 also includes a metal layer 212 disposed between the first and second isolation layers 214, 206. Portions 224 of the metal layer 212 are uncovered by the first isolation layer 214 to form metal contact pads 226. The first and second isolation layers 214, 206 may include polyimide. The metal layer 212 may include any one or more of titanium, gold and platinum.

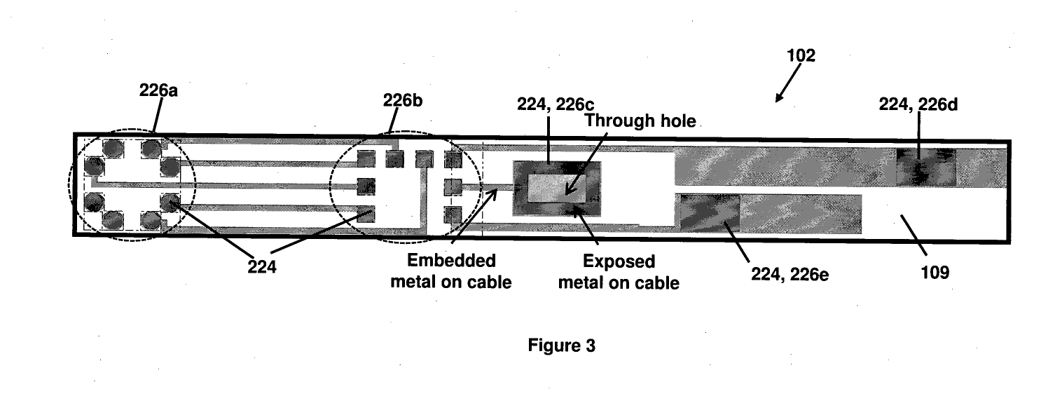

FIG. 3 shows a schematic top view of the strip 102. As shown in FIG. 3, the strip 102 has first to fifth metal contact pads 226a, 226b, 226c, 226d, 226e (e.g. uncovered portions 224 of the metal layer 212). The strip 102 also includes at least one through-hole 302 formed through the at least one metal contact pad 226 and the second isolation layer 204 (not shown). For illustration purposes, only one through-hole 302 is shown in FIG. 3. The through-hole 302 is formed through the third metal contact pad 226c and the second isolation layer 204 (not shown). In one embodiment, the through-hole 302 is a via.

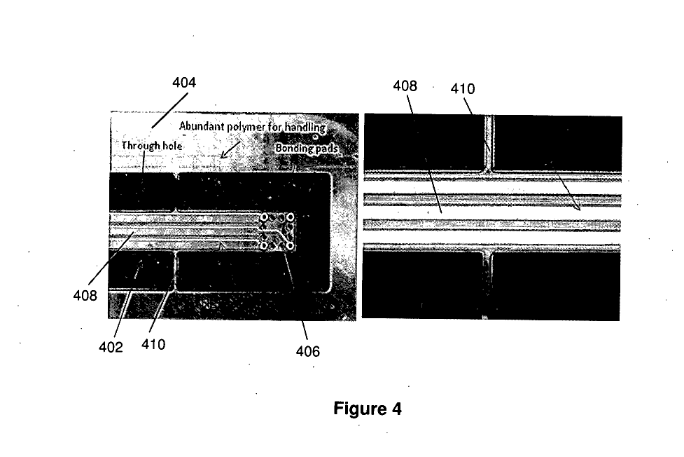

FIG. 4 shows images of a top view of the strip 102. The strip 102 has two parts, namely a center part 402 and an outer part 404. The center part 402 has bonding structure 406 for device bonding and metal traces 408 for electrical connection with metal wires. The bonding structure 406 and the metal traces 408 may correspond to e.g. the first to fifth metal contact pads 226a, 226b, 226c, 226d, 226e of FIG. 3. In one embodiment, the width of the center part 402 may be about 250 μm. The outer part 404 is connected with the center part 402 via thin polyimide traces 410. The thin polyimide traces 410 can be used as folding points (e.g. folding point 112 of FIG. 1b and further folding point 116 of FIG. 1c). The outer part 404 can be used for handling and can be easily torn off after assembly of e.g. devices and metal wires on the strip 102. In one embodiment, the width of the outer part 404 may be about 2 mm. Both the center part 402 and the outer part 404 of the strip 102 can ease the assembly process of components on the strip 102.

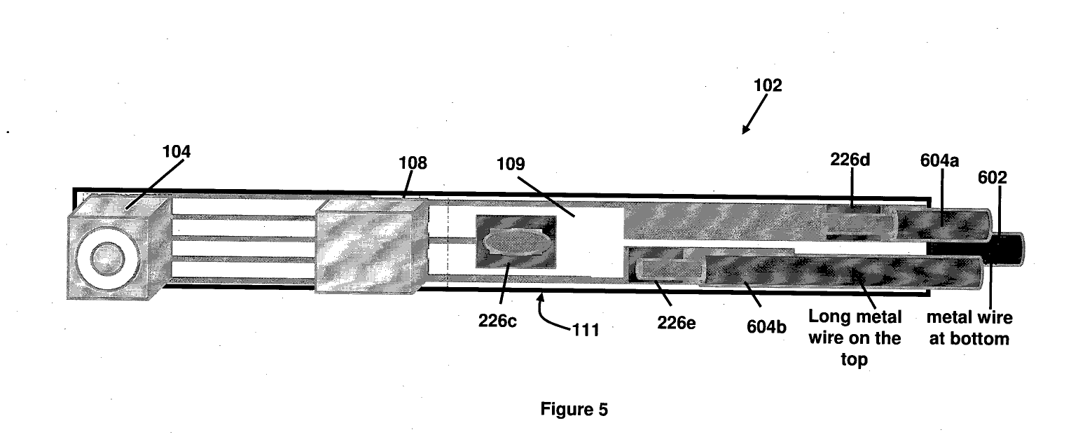

FIG. 5 shows that the sensor 104 and the chip 108 are disposed on the uncovered portions 224 of the metal layer 212 for electrically connecting the strip 102. The sensor 104 and the chip 108 are disposed on the first contact pad 226a (FIG. 3) and the second contact pad 226b (FIG. 3) respectively.

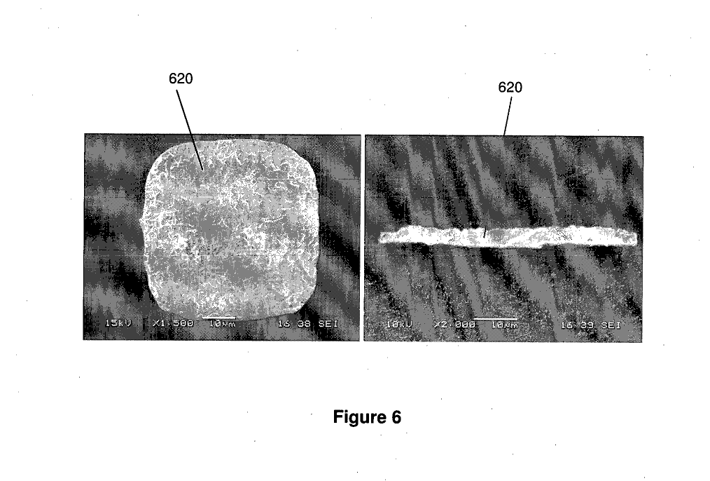

FIG. 6 shows images of a solder bump 620 formed on a test chip (not shown). In one embodiment, the solder bump 620 may have a 90 μm pitch and a diameter of about 40 μm. The test chip having a plurality of solder bumps 620 may be formed using the following exemplary process. An oxide layer and a silicon nitride (SiN) layer may be deposited on a wafer. A 7 μm negative resist coating may be deposited. Metal layers of Chronium (Cr) 200 A/Platinum (Pt) 5000 A/Tin (Sn) 5.5 μm/Platinum (Pt) 100 A may be deposited on the negative resist coating. After resist lift off, the test chips may be patterned. After wafer thinning and dicing, the test chip with a size of about 350 μm×350 μm and a height of about 400 μm may be obtained. In another embodiment, solder bumps 620 may be formed using a different method such as gold stud bumping using wire bonding equipment on e.g. a test chip aluminum pad.

As such, the sensor 104 and the chip 108 may have solder bumps (e.g. solder bumps 620 of FIG. 6) for electrical connections with the first contact pad 226a and the second contact pad 226b respectively.

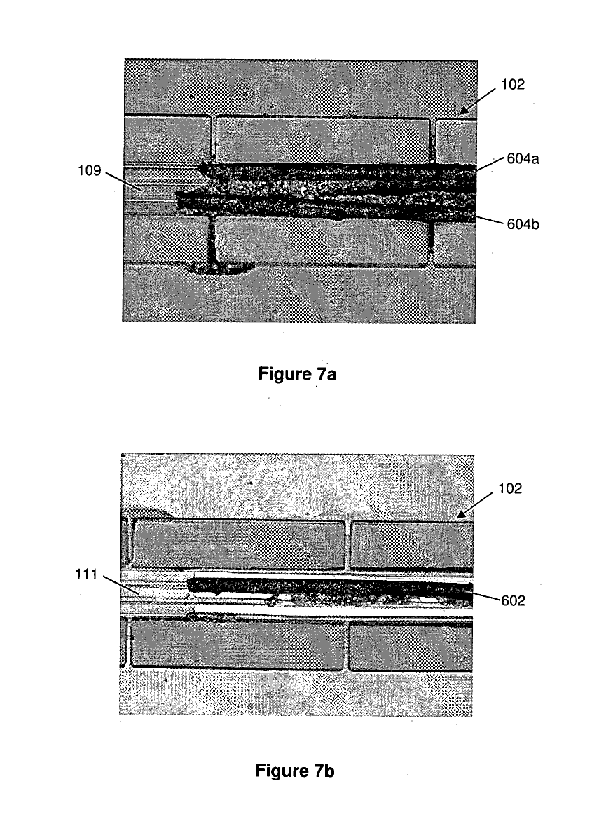

Referring back to FIG. 5, at least one first wire 602 is disposed on the second surface 111 of the strip 102 and is attached to the third metal contact pad 226c via the through-hole 302. For illustration purposes, only one first wire 602 is shown in FIG. 6. The first wire 602 extends through the through-hole 302. An image of the two second wires 604a, 604b disposed on the first surface 109 of the strip 102 is shown in FIG. 7a.

At least one second wire 604 is disposed on the first surface 109 of the strip 102. For illustration purposes, two second wires 604a, 604b are shown in FIG. 6. The two second wires 604a, 604b are attached to the fourth metal contact pad 226d and the fifth metal contact pad 226e respectively. An image of the first wire 602 disposed on the second surface 111 of the strip 102 is shown in FIG. 7b.

The first wire 602 and the two second wires 604a, 604b may be attached to the corresponding metal contact pads 226c, 226d, 226e using conductive glue or solder material. The solder material may include solder paste or solder alloys. Various materials can be used for the first wire 602 and the two second wires 604a, 604b. The first wire 602 and the two second wires 604a, 604b may include any one or more of aluminum, copper, titanium, tungsten, gold and silver.

The arrangement of disposing the first wire 602 on the second surface 111 of the strip 102 and disposing the two second wires 604a, 604b on the first surface 109 of the strip 102 can save space for the guide wire arrangement 100. The first wire 602 and the two second wires 604a, 604b can act as the core of the guide wire arrangement 100. Since no core element is used, a guide wire arrangement 100 having a small diameter can be obtained. For example, by using the first wire 602 and the two second wires 604a, 604b with a diameter of about 100 μm, a guide wire arrangement 100 having a diameter of about 350 μm or less can be obtained. The first wire 602 and the two second wires 604a, 604b can provide electrical connections and mechanical support for the guide wire arrangement 100. Further, the first wire 602 and the two second wires 604a, 604b with sub-millimeter diameter may have low resistance value.

In one embodiment, the strip 102 may be folded at the folding point 112 and the further folding point 116 after the sensor 104 and the chip 108 are attached to the strip 102 and before the first wire 602 and the two second wires 604a, 604b are attached to the strip 102. In another embodiment, the strip 102 may be folded at the folding point 112 and the further folding point 116 after the sensor 104, the chip 108, the first wire 602 and the two second wires 604a, 604b are attached to the strip 102.

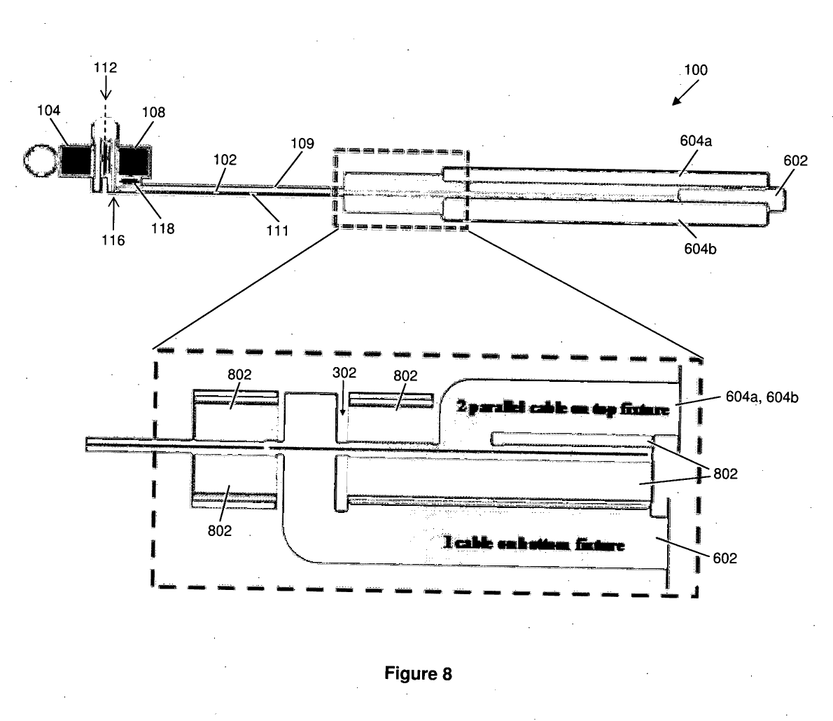

FIG. 8 shows that the guide wire arrangement 100 includes fixtures/holders 802 formed on the first surface 109 and the second surface 111 of the strip 102. The fixtures 802 form a guide for wire attachment to the strip 102. In other words, the fixtures 802 may be formed before the first wire 602 and the two second wires 604a, 604b are disposed on the strip 102. Various materials may be used to form the fixtures 802. The fixtures 802 may include silicon or polymer.

Using the fixtures 802 can enable wire attachment to be simpler, more reliable, and more manufacturable. If no fixtures are used, the two second wires 604a, 604b disposed on the first surface 109 of the strip 102 have to be parallel to prevent shorting between the two second wires 604a, 604b. The gap between the two second wires 604a, 604b is preferably about 40 μm. As such, the two second wires 604a, 604b may preferably be straight for a certain distance along the first surface 109 of the strip 102 (e.g. about 3-5 mm). However, it is difficult to do so without fixtures 802 when the wires have a small diameter and are soft. Therefore, by using fixtures 802, the attachment between the first wire 602 and the two second wires 604a, 604b and the strip 102 can be improved. Therefore, the guide wire arrangement 100 may have better manufacturability. The shorting between the two second wires 604a, 604b e.g. caused by overflow of conductive glue or solder material used to attach the two second wires 604a, 604b to the strip 102 can be prevented by using fixtures 802. Therefore, the guide wire arrangement 100 may have better reliability. The fixtures 802 can provide mechanical and electrical connection between the strip 102 and the wires 602, 604a, 604b.

Further, a non-conductive layer (not shown) may be deposited on the first wire 602 and the two second wires 604a, 604b. The non-conductive layer may be deposited using chemical vapor deposition, spray coating or dipping in molten polymer. Various materials may be used for the non-conductive layer. The non-conductive layer may include polymer or Parylene C. The non-conductive layer may be used for insulating the first wire 602 and the two second wires 604a, 604b. The two second wires 604a, 604b may be covered with the non-conductive layer to prevent short circuit. The non-conductive layer may have a thickness in a range of microns (μm).

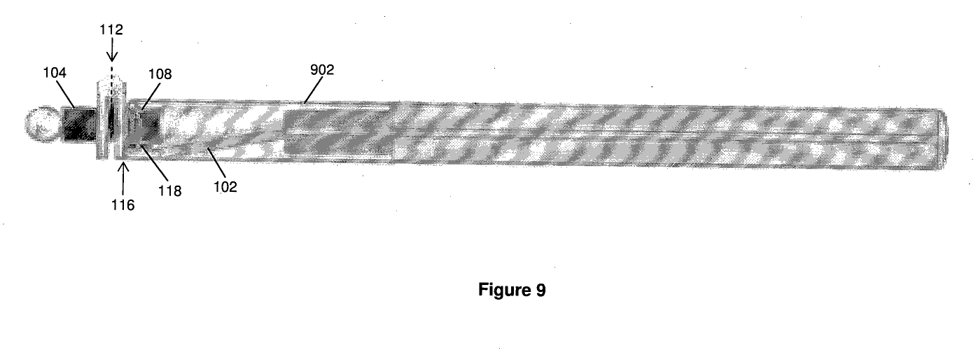

FIG. 9 shows that the guide wire arrangement 100 further includes a housing 902. A part of or whole of the strip 102 may be received in the housing 902. The housing 902 may be a plastic sleeve or tubing.

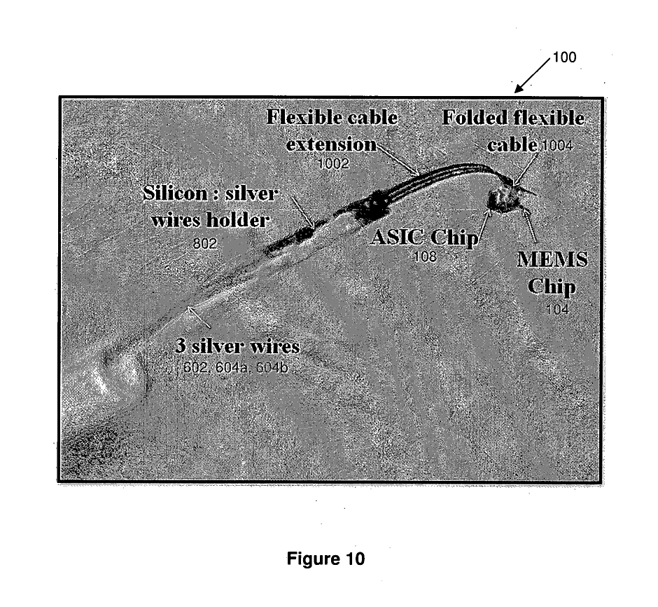

FIG. 10 shows an image of the guide wire arrangement 100. It can be seen from FIG. 10 that the discrete tiny components, e.g. the sensor 104, the chip 108, the wires 602, 604a, 604b with sub-millimeter diameter, and the fixtures/holders 802, are integrated on the strip 102 (e.g. a thin biocompatible flexible circuit). The strip 102 may have a flexible cable extension 1002 and a folded flexible portion 1004.

Since the strip 102 is thin and can be folded, the sensor 104 and the chip 108 can be placed side by side or can be stacked. The above description describes the sensor 104 and the chip 108 being arranged side by side on the strip 102 and the strip 102 being folded such that the sensor 104 and the chip 108 are stacked. Thus, the following description describes the sensor 104 and the chip 108 being arranged in a stack on the strip 102 without folding of the strip 102.

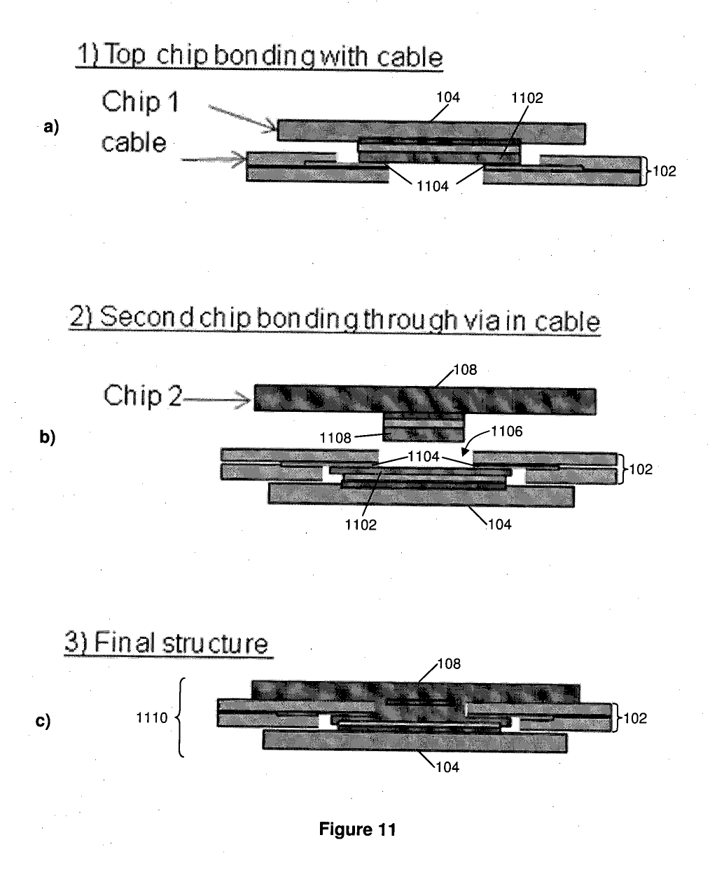

FIG. 11 shows an exemplary assembly process of arranging the sensor 104 and the chip 108 in a stack on the strip 102. FIG. 11a shows that solder bumps 1102 of the sensor 104 are aligned with a corresponding metal contact pad 1104 on the strip 102. The solder bumps 1102 of the sensor 104 are bonded to the metal contact pad 1104 of the strip 102 at about 270° C. e.g. using Flip Chip bonding machine. FIG. 11b shows that the strip 102 and the sensor 104 are flipped over. The chip 108 is bonded to the sensor 104 through a via 1106 in the strip 102. Solder bumps 1108 of the chip 108 are aligned with the solder bumps 1102 of the sensor 104 though the via 1106. Bonding of the chip 108 and the sensor 104 is performed at a reflow temperature. FIG. 11c shows a final structure 1110 of the sensor 104 and the chip 108 arranged in a stack on the strip 102.

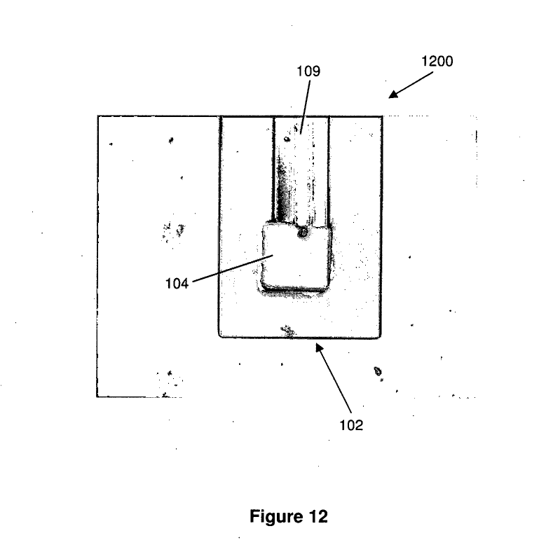

FIG. 12 shows an image of a top view of an arrangement 1200 having two dummy chips (e.g. the sensor 104 and the chip 108) respectively bonded on the top and bottom surfaces of the flexible circuit, i.e. on the first surface 109 and the second surface 111 of the strip 102. Only one dummy chip (e.g. the sensor) bonded on the first surface 109 of the strip 102 is shown in FIG. 12. The two dummy chips may have a size of about 350 μm×350 μm and a thickness of about 400 μm.

In one embodiment, the guide wire arrangement 100 may be a minimally invasive intra-vascular medical device. The guide wire arrangement 100 may be a sensorized guidewire which uses e.g. tactile sensor, pressure sensor, cochlea implants or image sensor. The guide wire arrangement 100 may be used as pacemaker leads.

The guide wire arrangement 100 can use folding of the strip 102 to achieve a vertical stack arrangement of the sensor 104 and the chip 108. The vertical stack arrangement of the sensor 104 and the chip 108 can enable miniaturization for the guide wire arrangement 100. Thus, the guide wire arrangement 100 having a small diameter can be achieved.

Further, the guide wire arrangement 100 can be formed using a simple and better manufacturability process. In other words, the strip 102, the sensor 104, the chip 108 and the wires 602, 604a, 604b can be integrated without complex process steps. For example, the sensor 104 may be easily mounted on the strip 102. Thus, lower costs may be incurred for manufacturing the guide wire arrangement 100.

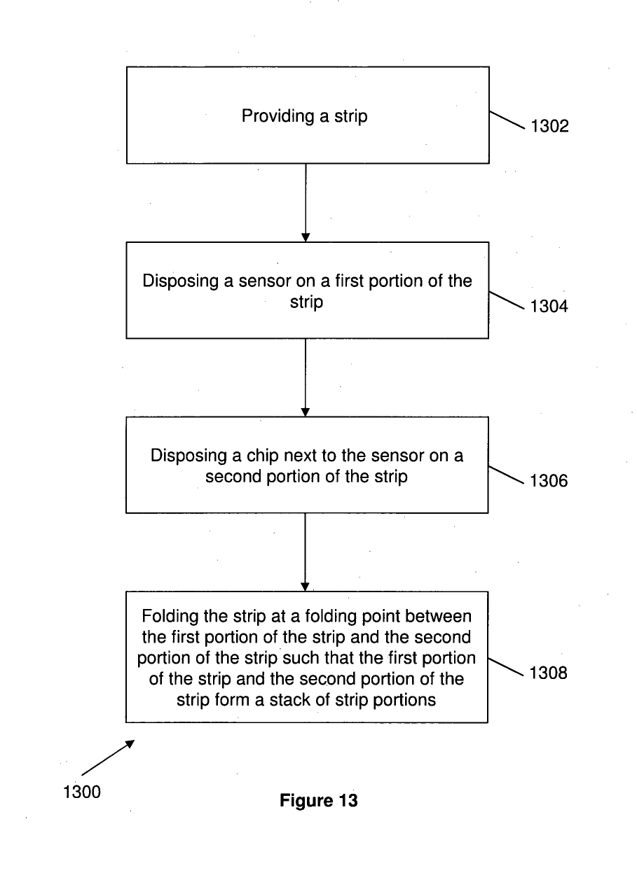

FIG. 13 shows a flowchart 1300 of a method of forming a guide wire arrangement. At 1302, a strip is provided. At 1304, a sensor is disposed on a first portion of the strip. At 1306, a chip is disposed next to the sensor on a second portion of the strip. The second portion of the strip may be next to the first portion of the strip. At 1308, the strip is folded at a folding point between the first portion of the strip and the second portion of the strip such that the first portion of the strip and the second portion of the strip form a stack of strip portions.

The strip may be folded at the folding point such that the sensor faces away from the second portion of the strip and the chip faces away from the first portion of the strip. The strip may be folded at the folding point by about 175 degrees to about 185 degrees. The method may further include folding the strip at a further folding point next to the chip and at the opposite side of the chip than the folding point such that the stack of strip portions is between the folding point and the further folding point. The strip may be folded at the further folding point by about 85 degrees to about 95 degrees.

The strip may include a first isolation layer providing a first surface of the strip, a second isolation layer providing a second surface of the strip, and a metal layer disposed between the first and second isolation layers. The method may further include removing portions of the first isolation layer to uncover portions of the metal layer. The uncovered portions of the metal layer may form metal contact pads. The sensor and the chip may be disposed on the uncovered portions of the metal layer for electrically connecting the strip.

The method may further include forming at least one through-hole through the at least one metal contact pad and the second isolation layer. The method may further include disposing at least one first wire on the second surface of the strip and attaching the at least one first wire to the at least one metal contact pad via the through-hole, and disposing at least one second wire on the first surface of the strip and attaching the at least one first wire to another metal contact pad. The at least one first wire and the at least one second wire may be attached to the corresponding metal contact pads using conductive glue or solder material.

The method may further include forming fixtures on the first surface and the second surface of the strip. The fixtures may form a guide for wire attachment to the strip. The method may further include depositing a non-conductive layer on the at least one first wire and the at least one second wire. The non-conductive layer may be deposited on the wires using any one of chemical vapor deposition, spray coating and dipping in molten polymer. The method may further include securing the folded areas of the strip using epoxy. The method may further include placing a part of or whole of the strip in a housing.

In one embodiment, the guide wire arrangement may have a strip in a form of e.g. a flexible cable. A sensor and a chip may be disposed at one end of the strip. In one embodiment, the sensor and the chip may be arranged adjacent to each other on a same surface of the strip. The strip may then be folded such that the sensor and the chip are in a stack arrangement. In another embodiment, the sensor and the chip may be arranged in a stack on the strip e.g. via a through-hole in the strip. The sensor and the chip may be attached to respective metal contact pads formed on the strip such that the sensor, the chip and the strip are electrically connected.

Further, wires may be disposed on the strip. At least one wire may be disposed on a first surface of the strip and at least one wire may be disposed on a second surface of the strip. The wires may be attached to respective metal contact pads formed on the strip such that the wires and the strip are electrically connected. At least one wire may be guided through e.g. a through-hole formed in the strip. Fixtures or holders may be formed on the strip to act as wire guiding structures. The fixtures or holders may also act as insulation structures between the wires to prevent shorting. In addition, an insulating or non-conductive layer may be disposed or deposited on the wires for insulation purposes. Thus, the guide wire arrangement may include the strip integrated with the sensor, the chip, the wires and the fixtures/holders. A part or the whole of the strip integrated with the sensor, the chip, the wires and the fixtures/holders may be received in a housing.

Therefore, a process of forming the guide wire arrangement may include either arranging a sensor and a chip on a same surface of a strip and folding the strip such that the sensor and the chip are in a stack arrangement or arranging the sensor and the chip in a stack arrangement on the strip. The process may also include disposing at least one wire on a first surface and a second surface of the strip respectively. The process may further include forming fixtures or holders on the strip. The process may further include disposing or depositing an insulating or non-conductive layer on the wires. The process may further include disposing a part or the whole of the strip integrated with the sensor, the chip, the wires and the fixtures/holders in a housing.

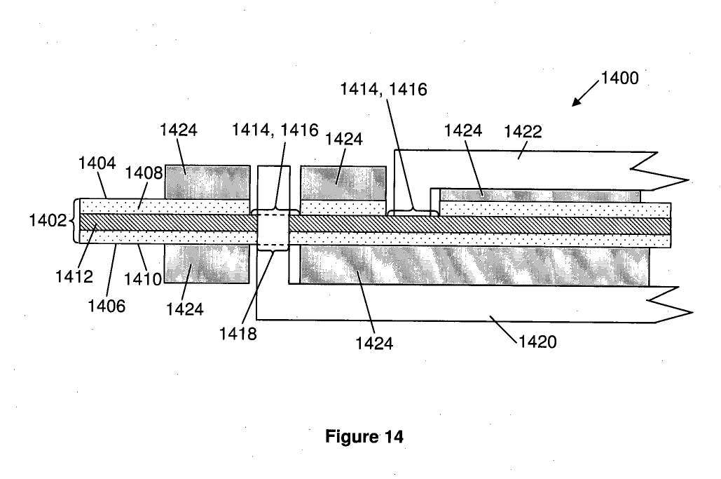

FIG. 14 shows a schematic diagram of a strip arrangement 1400. The strip arrangement 1400 includes a strip 1402 having a first surface 1404 and a second surface 1406. The strip 1402 includes a first isolation layer 1408 providing the first surface 1404 of the strip 1402, and a second isolation layer 1410 providing a second surface 1406 of the strip 1402. The strip 1402 also includes a metal layer 1412 disposed between the first isolation layer 1408 and the second isolation layer 1410. Portions 1414 of the metal layer 1412 are uncovered by the first isolation layer 1408 to form metal contact pads 1416. At least one through-hole 1418 is formed through the at least one metal contact pad 1416 and the second isolation layer 1410. The through-hole 1418 may be a via.

The strip arrangement 1400 includes at least one first wire 1420 disposed on the second surface 1406 of the strip 1402 and electrically connected to the strip 1402 via the through-hole 1418 formed in the strip 1402. The strip arrangement 1400 also includes at least one second wire 1422 disposed on the first surface 1404 of the strip 1402 and electrically connected to the strip 1402. The strip arrangement 1400 includes fixtures 1424 formed on the first surface 1404 and the second surface 1406 of the strip 1402. The fixtures 1424 may form a guide for wire attachment to the strip 1402.

In one embodiment, the strip arrangement 1400 may be a guide wire arrangement.

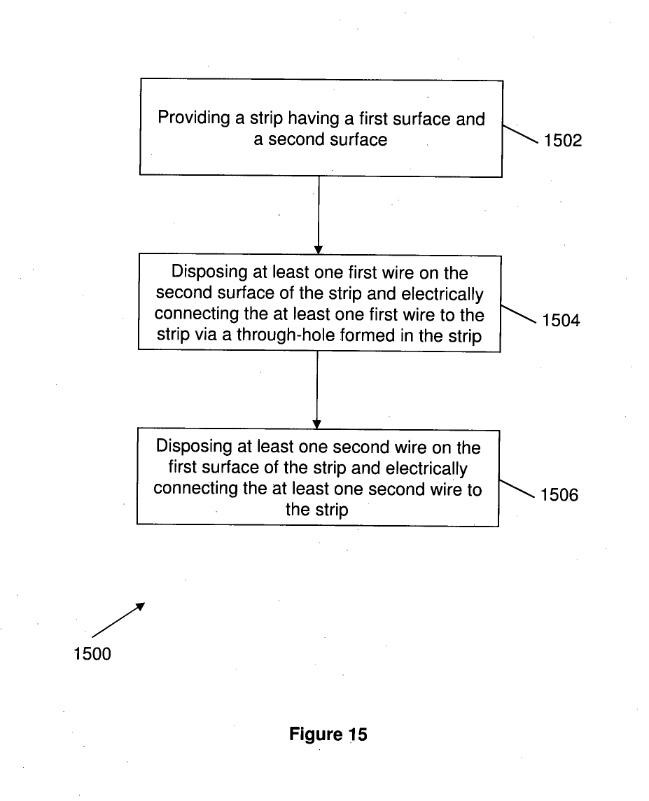

FIG. 15 shows a flowchart 1500 of a method of forming a strip arrangement. At 1502, a strip having a first surface and a second surface is provided. At 1504, at least one first wire is disposed on the second surface of the strip and the at least one first wire is electrically connected to the strip via a through-hole formed in the strip. At 1506, at least one second wire is disposed on the first surface of the strip and the at least one second wire is electrically connected to the strip.

The strip may include a first isolation layer providing a first surface of the strip, a second isolation layer providing a second surface of the strip, and a metal layer disposed between the first and second isolation layers. The method may further include removing portions of the first isolation layer to uncover portions of the metal layer. The method may further include forming the at least one through-hole through the at least one metal contact pad and the second isolation layer. The method may further include forming fixtures on the first surface and the second surface of the strip. The fixtures may form a guide for wire attachment to the strip.

In one embodiment, a strip arrangement may have a strip in a form of e.g. a flexible cable. The strip arrangement may have at least one wire disposed on a first surface of the strip, and at least one wire disposed on a second surface of the strip. The wires may be attached to respective metal contact pads formed on the strip such that the wires and the strip are electrically connected. At least one wire may be guided through e.g. a through-hole formed in the strip. The strip arrangement may have fixtures or holders formed on the strip to guide the placement of the wires on the strip. The fixtures or holders may also act as insulators disposed between the wires to prevent shorting. In addition, insulation or non-conductive materials may be disposed or deposited on the wires for insulation purposes.

Therefore, a process of forming the strip arrangement may include disposing at least one wire on a first surface and a second surface of the strip respectively. The process may further include forming fixtures or holders on the strip. The process may further include disposing or depositing insulation or non-conductive materials on the wires.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

In this document, the following documents are cited:
[1] Haga, Y. Mineta, T. Esashi, M. Yamagata, “Active catheter, active guidewire and related sensor systems” Automation Congress, 2002 Proceedings of the 5th Biannual World pp. 291-296, 2002
[2] Keith J. Rebello, “Applications of MEMS in Surgery”. Proceedings of the IEEE, Vol. 92, pp. 43-55, 2004
[3] Gianluca Bonanomi, Keith Rebello, Kyle Lebouitz, Cameron Riviere, Elena Di Martino, David Vorp, Marco A. Zenati, “Microelectromechanical systems for endoscopic cardiac Surgery”, J. Thorac Cardiovasc Surg, Vol. 126, pp. 851-852, 2003

1. A guide wire arrangement, comprising:
a strip;
a sensor being disposed on a first portion of the strip;
a chip being disposed next to the sensor on a second portion of the strip, wherein the second portion of the strip is next to the first portion of the strip;
wherein the strip is folded at a folding point between the first portion of the strip and the second portion of the strip such that the first portion of the strip and the second portion of the strip form a stack of strip portions.

2. The guide wire arrangement of claim 1,
wherein the strip is folded at the folding point such that the sensor faces away from the second portion of the strip and the chip faces away from the first portion of the strip.

3. The guide wire arrangement of claim 1,
wherein the strip is folded at the folding point by about 175 degrees to about 185 degrees.

4. The guide wire arrangement of claim 1,
wherein the strip is folded at a further folding point next to the chip and at the opposite side of the chip than the folding point such that the stack of strip portions is between the folding point and the further folding point.

5. The guide wire arrangement of claim 4,
wherein the strip is folded at the further folding point by about 85 degrees to about 95 degrees.

6. The guide wire arrangement of claim 1,
wherein the strip comprises:
a first isolation layer providing a first surface of the strip;
a second isolation layer providing a second surface of the strip;
a metal layer disposed between the first and second isolation layers;
wherein portions of the metal layer are uncovered by the first isolation layer to form metal contact pads.

7. (canceled)

8. (canceled)

9. The guide wire arrangement of claim 6,
wherein the sensor and the chip are disposed on the uncovered portions of the metal layer for electrically connecting the strip.

10. The guide wire arrangement of claim 6,
wherein the strip comprises at least one through-hole formed through the at least one metal contact pad and the second isolation layer.

11. The guide wire arrangement of claim 10, further comprising:
at least one first wire being disposed on the second surface of the strip and attached to the at least one metal contact pad via the through-hole;
at least one second wire being disposed on the first surface of the strip and attached to another metal contact pad.

12. The guide wire arrangement of claim 11,
wherein the at least one first wire extends through the through-hole.

13. (canceled)

14. The guide wire arrangement of claim 11,
wherein the at least one first wire and the at least one second wire are attached to the corresponding metal contact pads using conductive glue or solder material.

15. (canceled)

16. The guide wire arrangement of claim 11,
further comprising fixtures formed on the first surface and the second surface of the strip, wherein the fixtures form a guide for wire attachment to the strip.

17. (canceled)

18. The guide wire arrangement of claim 11,
further comprising a non-conductive layer being deposited on the at least one first wire and the at least one second wire.

19. (canceled)

20. (canceled)

21. The guide wire arrangement of claim 1,
wherein the sensor comprises a microelectromechanical system sensor.

22. The guide wire arrangement of claim 1,
wherein the sensor is any one of a group consisting of a force sensor, a pressure sensor, a temperature sensor, an acceleration sensor, an angular velocity sensor, an electronic compass, and an ultrasound sensor.

23. (canceled)

24. The guide wire arrangement of claim 1,
wherein the folded areas of the strip are secured using epoxy.

25. The guide wire arrangement of claim 1,
further comprising a housing, wherein a part of or whole of the strip is received in the housing.

26. (canceled)

27. The guide wire arrangement of claim 1,
wherein the guide wire arrangement is a minimally invasive intra-vascular medical device.

28. A method of forming a guide wire arrangement, the method comprising:
providing a strip;
disposing a sensor on a first portion of the strip;
disposing a chip next to the sensor on a second portion of the strip, wherein the second portion of the strip is next to the first portion of the strip;
folding the strip at a folding point between the first portion of the strip and the second portion of the strip such that the first portion of the strip and the second portion of the strip form a stack of strip portions.

29-42. (canceled)

43. A strip arrangement, comprising:
a strip having a first surface and a second surface;
at least one first wire being disposed on the second surface of the strip and electrically connected to the strip via a through-hole formed in the strip;
at least one second wire being disposed on the first surface of the strip and electrically connected to the strip.

44-53. (canceled)
